SEQUENCE LISTING (3 sequences)

SEQ ID NO: 1 - DNA, 23 bp, synthetic construct
  misc_feature 1..23: note = Probe P1
  source 1..23: mol_type = other DNA
gacgtctaaacctactaaagagg

SEQ ID NO: 2 - DNA, 25 bp, synthetic construct
  misc_feature 1..25: note = Probe P2
  source 1..25: mol_type = other DNA
ccttgtgtggtctgcatgagtttag

SEQ ID NO: 3 - DNA, 30 bp, synthetic construct
  misc_feature 1..30: note = Probe P3
  source 1..30: mol_type = other DNA
taacgttgttaggtactcgtcacgactgag

(19) United States
(12) Patent Application Publication
(10) Pub. No.: US 2025/0207181 A1
(43) Pub. Date: Jun. 26, 2025

(54) PROCESS FOR DETECTING A TARGET NUCLEIC ACID

(71) Applicants: ALMA MATER STUDIORUM - Universita' di Bologna (90%), BOLOGNA (IT); PERSONAL GENOMICS S.R.L. 10%), Verona (VR) (IT)

(72) Inventors: Luca Prodi, Bologna (BO) (IT); Sabrina Conoci, Bologna (BO) (IT); Giovanni Valenti, Bologna (BO) (IT); Francesco Paolucci, Bologna (BO) (IT); Paolo Garagnani, Bologna (BO) (IT); Katarzyna Malgorzata Kwiatkowska, Bologna (BO) (IT); Stefania Mariani, Verona (VR) (IT); Luciano Xumerle, Verona (VR) (IT)

(73) Assignees: ALMA MATER STUDIORUM - Universita' di Bologna (90%), BOLOGNA (IT); PERSONAL GENOMICS S.R.L. (10%), Verona (VR) (IT)

(21) Appl. No.: 18/851,095

(22) PCT Filed: Mar. 27, 2023
(86) PCT No.: PCT/IB2023/053007

§ 371 Date: Sep. 26, 2024

(30) Foreign Application Priority Data
Mar. 30, 2022 (IT) 102022000006245

(51) Int. Cl. C12Q 1/6825; C12Q 1/70

(52) U.S. Cl. CPC C12Q 1/6825; C12Q 1/701; C12Q 2600/156

(57) ABSTRACT

The present invention relates to a process for detecting an analyte in an isolated sample, wherein said analyte is a target nucleic acid, comprising the steps of: supplying a sample comprising at least one nucleic acid; heat-treating said sample; contacting the heat-treated sample with at least two single-stranded nucleic acid probes in which each of said at least two single-stranded nucleic acid probes is complementary to a corresponding portion of said target nucleic acid; adding an active luminophore; determining a luminescence signal generated by the active luminophore.

### TECHNICAL FIELD

The present invention relates to a process for rapid and sensitive detection of a nucleic acid, for example a whole genome, through the application of, among others, luminescence techniques such as, for example, photoluminescence and electrochemiluminescence (ECL). In particular, the process according to the invention combines a cooperative hybridization step of a target nucleic acid on the surface of an electrode derivatized with specific probes, with a detection step by electrochemiluminescence (ECL). The process allows direct detection of, for example, a whole target genome without any amplification of the analyte. Therefore, the process of the invention may be considered to represent an amplification-free approach, particularly a PCR-free approach. Furthermore, the process according to the present invention provides for a preliminary step of heat-treatment of the sample to be analyzed, which is much simplified compared to what is required according to known PRC-based methods.

### STATE OF THE ART

Molecular analysis of the nucleic acids (DNA and RNA) is crucial in many medical fields today for an early and accurate diagnosis, personalized therapy and preventive screening. This is particularly relevant in the field of infectious diseases, as became evident after the pandemic COVID-19 infection that affected more than 190 million people worldwide with more than 4 million deaths. Such urgency is particularly evident, in general, for countries where these diseases catastrophically affect the health of the population and particularly for developing countries.

Current analytical methodologies for the detection of, for example, COVID-19 are based on two main technologies: rapid tests for the immunochemical detection of SARS-Cov2 and molecular tests in the laboratory (so-called molecular swabs), based on the detection of viral genomic sequences by using PCR-based technologies.

The first type of tests, although rapid, nevertheless has limited sensitivity and generates false negatives, as is to date recognized by the international scientific community, a factor that delimits their effectiveness as a screening tool.

The second type of tests, also known in the case of COVID 19, as molecular swab, is a fairly complex laboratory method since it requires several analytical steps such as (a) extraction of the nucleic acid from the sample through specific kits and (b) detection through Real Time PCR (Polymerase Chain Reaction), itself also an inherently rather laborious and expensive method (involving several analytical and biochemical steps that must be carried out by experienced personnel) and, consequently, currently performed exclusively in specialized and centralized laboratories. These PCR-based methods involving amplification steps also make the nucleic acid quantification not easy, since they require internal standards for calibration.

Furthermore, known methods of molecular analysis generally involve a step of extracting nucleic acids from the biological sample collected. Such extraction methods are, in turn, complex to carry out. In fact, although special kits for the nucleic acid extraction are commercially available, such extraction requires skilled labor and long time to be accomplished.

Therefore, although current PCR-based methods are well-established methods, such methods are not suitable for use by unskilled personnel, close to the patient, and at competitive cost and time. This aspect is a strong limitation for its massive use, effectively limiting the potential of the molecular analysis for human health. Evidence of this is the current difficulty in obtaining massive screening and real-time diagnosis of COVID-19 for timely management and prevention of infections (molecular analysis by PCR takes at least 5 hours). Therefore, the development of new molecular methods that enable rapid and sensitive as well as reliable detection of pathogens is a significant breakthrough in the field of molecular diagnostics.

Such methods would enable the development of the so-called Point-Of-Care Technologies (POCT), which are the new frontier for medical diagnostics. These are bio-engineered systems integrated with innovative biotechnology, capable of rapid diagnostic testing in non-laboratory settings, performed by personnel who are not necessarily specialists or even by the patient himself. The ever-increasing interest in research in this area is largely led by two distinct trends in the modern society, namely the general need to reduce high health care costs and the demand for improved analytical solutions for early diagnosis and customized therapies.

The elimination of the amplification process (in particular, free of a PCR step) would be, as well as the simplification and integration of the nucleic acid extraction step, particularly advantageous for the development of systems of advanced molecular diagnostics by point-of-care (PoC) nucleic acid detection, as it would allow a major simplification of the method, resulting in rapid analysis and reduced costs.

### Objects of the Invention

Object of the present invention is to provide a process for detecting nucleic acids that enables direct, rapid and sensitive detection of nucleic acids, including whole genomes, from isolated samples, particularly isolated clinical samples.

Further object of the present invention is to provide a process for detecting nucleic acids that enables a sample to be analyzed immediately after its collection, and the result of such analysis to be obtained in a short time.

Object of the present invention is still to provide a process for detecting nucleic acids that is sensitive and reliable and that allows the possibility of false negatives to be minimized.

In addition, object of the present invention is to provide a process for detecting nucleic acids that may be performed with fully portable instrumentation and by unskilled personnel, including the patient himself.

Furthermore, object of the present invention is to provide a process for detecting nucleic acids that may be performed by involving extremely limited use of reagents and equipment made of plastic materials, and is therefore particularly environmentally sustainable.

Still an object of the present invention is to provide a kit for quickly and reliably detecting a target nucleic acid.

### DESCRIPTION OF THE INVENTION

The objects set forth above, as well as other objects, are achieved by the subject matter of the present invention, namely by a process for extracting and detecting an analyte in an isolated sample, in which said analyte is a target nucleic acid, characterized by a specific nucleotide sequence, said process comprising the steps of:
a) providing an isolated sample comprising at least one nucleic acid;
b) heat-treating said isolated sample, thus obtaining a heat-treated sample;
c) contacting said heat-treated sample with at least two single-stranded nucleic acid probes, each of such single-stranded nucleic acid probes being complementary to a corresponding portion of the target nucleic acid;
d) adding an active luminophore;
e) determining a luminescence signal generated by said active luminophore.

According to the present invention, said isolated sample is heat-treated, for example at a temperature between 95° C. and 98° C. over a period between 4 min and 7 min and subsequently cooled.

Still according to the present invention, said nucleic acid probes are selected from DNA, RNA, PNA.

Yet again according to the present invention, said luminescence signal may be a signal of photoluminescence, such as fluorescence or phosphorescence, chemiluminescence, thermo-chemiluminescence or electrochemiluminescence (ECL). Particularly preferred are photoluminescence or electrochemiluminescence (ECL) signals.

By the term “active luminophore” is meant herein to refer to a luminophore capable of giving a luminescence signal, particularly a signal of photoluminescence (for example, fluorescence or phosphorescence) and/or chemiluminescence and/or thermochemiluminescence and/or electrochemiluminescence (ECL). Preferably, the active luminophore is a luminophore capable of giving a photoluminescence and/or electrochemiluminescence (ECL) signal.

Surprisingly, it has been observed that the process of the invention allows complete molecular genetic analysis (extraction and detection) to be performed without the use of the PCR amplification technique, enabling rapid and sensitive detection of a target nucleic acid (even whole genomes) from an isolated sample. Specifically and according to a preferred embodiment of the present invention, the process combines a step of extracting the target nucleic acid and cooperatively hybridizing it (thus selectively immobilizing it on the surface of an electrode derivatized with specific probes) with a detection step based, for example, on electrochemiluminescence (ECL). The process allows direct detection of, for example, a whole target genome of an organism, in particular the genome of a pathogen, without any amplification of the analyte. Therefore, the process of the invention may be an amplification-free approach, particularly a PCR-free approach. Specifically, as mentioned above, the process of the invention is based on the combination of surface cooperative hybridization (SCH) of a target nucleic acid (for example, a whole target genome) by complementary oligo-ss (“single strand”) probes, specifically DNA, RNA or PNA, which are anchored on the surface of an ECL electrode, in the case of using the electrochemiluminescence technique as the detection system. Advantageously, these probes are designed to recognize specific sequences, for example, portions of specific sequences within the whole target genome. Advantageously, such probes are able to ensure simultaneous hybridization with the target nucleic acid (for example, when the target nucleic acid is a target genome, a sequence gap in the target genome may be maintained between the two probes). Upon hybridization, the target nucleic acid is recognized by the surface probes that independently hybridize the complementary strands of the target nucleic acid, anchoring it, for example, to the electrode surface and forming a supramolecular complex.

Advantageously, the process of the invention is very sensitive and is capable of meeting the requirements of Limit of Detection (LoD of 10 copies of target/reaction). The process of the invention may also be easily integrated into low-cost, portable devices, thus enabling its widespread use for diagnostic screening. Therefore, it enables the creation of inexpensive Point of Care tests (PoCTs).

Furthermore, the particular speed at which the process of the invention is carried out is aided by the presence of a step in which the biological sample containing the nucleic acid is heat-treated, for example, at a temperature between 95° C. and 98° C. over a period between 4 min and 7 min, and then cooled.

Such heat-treatment dispenses with the step of extracting the nucleic acid from the sample. In fact, such an extracting step requires long lead times and the use of skilled labor. According to what is known in the art, the extraction of the nucleic acid (DNA or RNA) from a sample is carried out by using appropriate, commercially available extraction kits.

According to an aspect, the process of the invention comprises the step of providing an isolated sample comprising at least one nucleic acid.

In embodiments of the invention, such a sample is a sample comprising biological material, itself comprising at least one nucleic acid.

In embodiments, the sample is selected from salivary swab, naso-oro-pharyngeal swab, mouth swab, vaginal swab, as well as isolated samples of biological fluids such as, for example, urine, blood, cerebrospinal fluid.

For example, in the case of airway infections (for example, SARS-Cov2 infections), the biological material comes from samples collected from the upper respiratory tract, for example, from subjects undergoing the COVID-19 diagnostic test.

In this specific case, the procedure, in general, consists of taking mucus lining the superficial cells of the mucosa of the nasopharynx or oropharynx, or a salivary sample, such as for example by swabbing. Sampling is done in few seconds, for example, by trained and specialized personnel who must ensure that the procedure is carried out correctly, avoiding both contamination of the sample and collection of only the outermost mucosal tract of the nasal pits, which would invalidate the result of the molecular test. For reasons of containment from potential infection, the procedure must preferably be carried out in a sterile environment by personnel provided with appropriate disposable personal protective equipment: gloves, gown, cap, mask, goggles or face shield. Sampling is performed by the use of the sampling kit comprised of, for example, a nasal swab made of synthetic material with a plastic rod and a test tube (Dnase/Rnase free) with a screw cap, containing an inactivating liquid transport medium which is stable at room temperature, suitable for the collection, transport and storage of pathogens. According to currently known techniques, collected samples are immediately transported to the laboratory or alternatively are stored in a refrigerator (+4° C.) for a period of time of less than 48 hours.

Still according to the invention, in the case described above, the sampling may also be performed by the same person who needs the diagnostic testing of COVID-19 or by third parties who are also unqualified. Advantageously, the process of the invention may be carried out from a saliva sample.

In embodiments, the target nucleic acid is DNA or RNA.

In embodiments, the target nucleic acid is the whole genome of an organism or virus.

In embodiments, the whole target genome may be single-stranded (ss-) or double-stranded (ds).

In embodiments, the target genome may be a sequence of the whole genome or a single-stranded (ss-) or double-stranded (ds) transcript thereof.

According to an aspect, the process according to the present invention comprises the step of heat-treating the sample at a temperature between 60° C. and 99° C. over a period between 1 min and 20 min and subsequently cooling.

As mentioned above, such a step of heat-treating the sample collected allows the extraction step of the nucleic acid to be avoided, thus reducing both the time and complexity of carrying out the process.

Preferably, the sample is treated at a temperature between 95° C. and 98° C. over a period between 1 min and 10 min, preferably between 4 min and 7 min, and subsequently cooled.

As mentioned above, the process of the invention is based on luminescence, which may be defined as the phenomenon related to the emission of light radiation by a body resulting from electron transitions from a higher energy level to another at lower energy. The phenomenon of luminescence may be caused, for example, by biochemical processes (bioluminescence), chemical reactions (chemiluminescence), electrochemical reactions (electrochemiluminescence—ECL), reactions induced by an increase in temperature (thermochemiluminescence), electrical phenomena or electrical charges (electroluminescence), the action of photons (fluorescence and phosphorescence) and nuclear radiation (radioluminescence).

According to a preferred aspect of the present invention, the process of the invention is based on the electrochemiluminescence (ECL) technique, which is the analytical technique based on a luminescent phenomenon induced by an electrochemical stimulus. In particular, with the ECL co-reagent mechanism, the excited state of a luminophore is generated by the reaction between luminophores and reactive intermediates of the co-reagent, which are produced by electrochemical oxidation or reduction. In particular, the process of the invention, in this particular embodiment, is based on molecular recognition of nucleic acid molecules by hybridization with probes anchored to the electrode surface and detection by ECL luminophore. The ECL signal increases in the presence of the target because the luminophore intercalates into the target once it has been recognized on the electrode surface.

According to an aspect, the process of the invention comprises the step of contacting the heat-treated sample with at least two single-stranded nucleic acid probes, wherein each of such single-stranded nucleic acid probes is complementary to a corresponding portion of the target nucleic acid.

In embodiments, the single-stranded nucleic acid probes are DNA, RNA or PNA.

According to the present description, the terms “DNA,” “RNA,” and “PNA” denote “deoxyribonucleic acid,” “ribonucleic acid,” and “peptide nucleic acid,” respectively.

In embodiments, the single-stranded nucleic acid probes are immobilized at an electrode, preferably through a surface linker.

In embodiments, the surface linker is HS—(CH₂)₆—.

Other surface linkers that may be used depend on the material by which the electrode is constructed. For example, HS—(CH₂)₆— linker is suitable for metals, while silanes of the (OR)₃—Si—(CH₂)₆— type may be used for carbon or graphene electrodes.

In embodiments, the electrode is an electrode made of gold, platinum, silver, graphene, glassy carbon, ITO (Indium Tin Oxide), which are possibly enriched with metal- or metal oxide-based nanostructures.

Preferably such an electrode is miniaturized.

The ECL-based process, according to a preferred aspect of the present invention, enables nucleic acid diagnosis and quantification, based on the combination of immobilization of the target nucleic acid on the surface of an electrode and the transduction of ECL by the ECL luminophore intercalating inside the double strand of the nucleic acid that is formed as a result of the hybridization of the target nucleic acid with the single-stranded probes.

According to an aspect, the process comprises the step of adding an active electrochemiluminescent luminophore, and the step of determining a luminescence signal generated by the active electrochemiluminescent luminophore.

In general, the intercalating electrochemiluminescent luminophore (ECL) is an intercalating agent, which shows high sensitivity, both in terms of luminescence duration and quantum yield. The ECL active molecule (alternatively referred to as ECL active luminophore or active ECL) is preferably a complex of Ru(II), Ir(III), Re(I), or Os(II), or biostructures or nanostructures derivatized with such complexes, more preferably a Ru(II) complex. Preferably, the ECL active luminophore is a Ru(II), Ir(III), Re(I) or Os(II) coordination complex.

In embodiments, the active electrochemiluminescent luminophore is a Ru(II), Ir(III), Re(I), or Os(II) coordination complex, preferably a Ru(II) coordination complex. Alternatively, organic fluorophores may be used.

In embodiments, when the whole target genome is a double-stranded genome, at least one of the at least two single-stranded nucleic acid probes is complementary to a portion of the parallel strand of the double-stranded genome, and at least another of the at least two probes is complementary to a portion of the anti-parallel strand of said whole double-stranded genome.

The two strands of a double-stranded genome are called “anti-parallel” because they are oriented in two opposite directions. The different orientation of the two strands may be identified by taking into account the free terminal groups (i.e., not bound to another nucleotide) which are located at the end of each strand (i.e., each polynucleotide chain). Each chain has at one end, called the 5′ end, a 5′ phosphate group (—OPO₃⁻) and at the other end, called the 3′ end, a hydroxyl group (—OH). In a double helix, for example, of DNA, the 5′ end of one strand corresponds to the 3′ end of the other strand; in other words, if an arrow is drawn from 5′ to 3′ for each strand, the two arrows point in opposite directions. For example, the strand running in the 5′ to 3′ direction may be called a “parallel strand”, whereas its complementary strand running in the 3′ to 5′ direction may be called an “anti-parallel strand”.

In embodiments, the nucleic acid, for example, the target genome, is the nucleic acid of an organism selected from viruses, bacteria, parasites or eukaryotic cells.

In embodiments, the nucleic acid is the nucleic acid of a SARS-Cov2 virus.

Preferably, the nucleic acid is the whole genome of the SARS-Cov2 virus.

In embodiments, when the whole target genome is the SARS-Cov2 genome, the single-stranded nucleic acid probes comprise at least the sequence GACGTCTAAACCTACTAAAGAGG (SEQ. ID. NO. 1), the sequence CCTTGTGTGGTCTGCATGAGTTTAG (SEQ. ID. NO. 2) and the sequence TAACGTTGTTAGGTACTCGTCACGACTGAG (SEQ. ID. NO. 3). Preferably, such probes are immobilized at an electrode, preferably by a surface linker. In embodiments, the linker may be HS—(CH₂)₆—.

Advantageously, the single-stranded probes may be designed so to selectively recognize different variants of the same pathogen.

For example, the process of the invention may involve the use of probes which allows to detect the pathogen genome and recognize its variant.

In embodiments, the active electrochemiluminescent luminophore has a ligand selected from dppz (dipyrido[3,2-a:2′,3′-c]phenazine), 1,10-phenanthroline, 2,2′-bipyridile quinoxalino[2,3-f][1,10]phenanthroline.

In embodiments, the ECL active luminophore has a dppz binder. Preferably, the active ECL luminophore is a Ru(II) complex with dppz ligand (dppz=dipyrido[3,2-a:2′,3′-c]phenazine), preferably Ru(bpy)₂dppz]²⁺ or [Ru(phen)₂dppz]²⁺ (wherein phen=1,10-phenanthroline, bpy=2,2′-bipyridine).

In embodiments, preferably, the active electrochemiluminescent luminophore is [Ru(bpy)₂dppz]²⁺ or [Ru(phen)₂dppz]²⁺, wherein phen=1,10-phenanthroline, bpy=2,2′-bipyridine, and dppz=dipyrido[3,2-a:2′,3′-c]phenazine.

In embodiments, the luminescent signal is generated electrochemically by using one or more sacrificial coreagents.

In embodiments, the ECL coreagent is a sacrificial reagent that, after oxidation or reduction, is capable of generating reactive radicals for ECL signal generation.

In embodiments, one or more sacrificial coreagents are selected from S₂O₈²⁻ (peroxydisulfate ion), C₂O₄²⁻ (oxalate ion), tertiary amines, tri-n-propylamine, 2-(dibutylamino)ethanol and hydrogen peroxide.

A further object of the present invention is a kit for the detection of at least one target nucleic acid.

According to the present invention, the kit comprises a working electrode comprising an appropriate material suitable for functionalization, a reference electrode and a counter electrode (or auxiliary electrode).

For example, the working electrode is an electrode made of gold, platinum, silver, graphene, glassy carbon, ITO (Indium Tin Oxide), which are possibly enriched with metal- or metal oxide-based nanostructures.

Preferably, the working electrode is functionalized with at least two single-stranded nucleic acid probes, each of such single-stranded nucleic acid probes being complementary to a corresponding portion of a target nucleic acid.

Preferably such an electrode is miniaturized.

For example, the reference electrode may be an Ag/AgCl electrode.

In embodiments, the kit further comprises a system for inlet and outlet flow control and/or a system for heating.

In embodiments, it comprises a working electrode comprising an appropriate material suitable for functionalization, a reference electrode and a counter electrode (or auxiliary electrode) and, optionally, an inlet and outlet flow control system and/or a heating system are integrated into a single device.

Advantageously, in an embodiment, a plurality of electrodes may be used, each equipped with probes designed to identify different nucleic acids (for example, genomes of different pathogens) in order to enable a plurality of diagnostic tests simultaneously.

For example, in embodiments, a plurality (an array) of electrodes may be used, each equipped with probes adapted to identify different variants of the same pathogen (for example, different variants of SARS-Cov2). Advantageously, in this case, both the actual presence of the pathogen and its specific variant may be determined in a single analysis.

The invention may be even better understood thanks to the illustrative, non-limiting examples described in the following Experimental Section and accompanied by the FIGS. 1-5.

### BRIEF DESCRIPTION OF THE FIGURES

FIG. 1 schematically shows an embodiment of the process, in which the following steps are highlighted: (a) Immobilization of specific probes (P1, P2, and P3) on the surface of the gold electrode (Au), by incubation for 4 hours; (b) immobilization of thiol (HSC₆H₁₂OH) on the surface of the gold electrode, by incubation overnight (c); recognition of the RNA genome of SARS-Cov2 (COVID 19) and hybridization with said genome by the probes and formation of the supramolecular complex, by incubation for 3-4 hours at 50° C.; (d) intercalation of [Ru(phen)₂dppz]²⁺, by incubation for 2 hours, and quantification of the RNA of SARS-Cov2 (COVID 19).

FIG. 2 depicts two graphs. In a first graph, FIG. 2(a), we show the electrochemiluminescence intensity versus the potential for different concentration of the synthetic genome of SARS-Cov2 (COVID 19) 0-350000 cps/ml; FIG. 2(b) shows a graph depicting the electrochemiluminescence intensity versus the concentration of COVID 19 (SARS-Cov2) in 0.1 M and 50 mM K₂S₂O₈ phosphate buffer. Working electrode vs. reference electrode Ag/AgCl with a scan rate of 0.3 V/s. PMT polarization of 750 V.

FIG. 3 shows the ECL intensity vs. potential for 4 genome samples of SARS-Cov2 (COVID 19), which are extracted from real samples, of which three tested positive and one negative (60937, white) to molecular swab. Gold working electrode and the potential is specified relative to the Ag/AgCl reference electrode with 0.3 V/s scan rate in 0.1 M and 50 mM K₂S₂O₈ phosphate buffer.

FIG. 4 shows the ECL intensity vs. potential for genome samples of SARS-Cov2 (COVID 19), which are obtained from real samples, in which the extraction step of the RNA genome was replaced with three different types of treatment: i) heat-treatment (HT), ii) proteinase K (PK) hydrolysis, and iii) proteinase hydrolysis and heat-treatment in combination (PK+HT) iv) without extraction (DIR, control). Gold working electrode and the potential is specified relative to the Ag/AgCl reference electrode with 0.3 V/s scan rate in 0.1 M and 50 mM K₂S₂O₈ phosphate buffer.

FIG. 5 shows a comparison between the results obtained by heat treating the sample and those obtained by extracting the nucleic acid by known techniques.

### EXPERIMENTAL SECTION

### Example 1—Probes for SARS-Cov2 and Genome Detection Conditions for SARS-Cov2

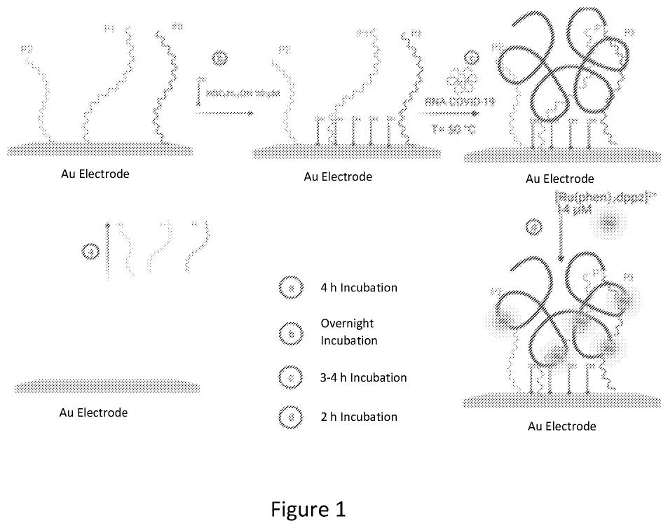

For the quantification of the RNA genome of SARS-Cov2, a process was developed that combines surface anchoring of the whole target genome by three single-stranded probes (P1, P2 and P3 described below) with the ECL transduction mechanism, as shown in FIG. 1.

### Probe P1

Sequence 5′-3′ name:
(SEQ. ID. NO. 1)
Cov-FW Thiol-C6-GACGTCTAAACCTACTAAAGAGG

Oligo ID: 210712X049B02 1/3
BC: 23
Purification: Desalinated
EC: 235
MW (Da): 7,394
Tm (melting temperature) (° C.): 61
OD (optical density) 260 nm: 25.7
Nmol: 109.5
μl needed for 100 μM solution: 1,095

### Probe P2

Sequence 5′-3′ name:
(SEQ. ID. NO. 2)
Cov-Rev Thiol-C6-CCTTGTGTGGTCTGCATGAGTTTAG

Oligo ID: 210712X049B03 2/3
BC: 25
Purification: Desalinated
EC: 233
MW (Da): 8,038
Tm (melting temperature) (° C.): 66
OD (optical density) 260 nm: 25.0
Nmol: 107.3
μl needed for 100 μM solution: 1,073

### Probe P3

Sequence 5′-3′ name:
(SEQ. ID. NO. 3)
Cov-Prob Thiol-C6-TAACGTTGTTAGGTACTCGTCACGACTGAG

Oligo ID: 210712X049B04 3/3
BC: 30
Purification: Desalinated
EC: 292
MW (Da): 9,565
Tm (melting temperature) (° C.): 71
OD (optical density) 260 nm: 28.0
Nmol: 96.0
μl needed for 100 μM solution: 960

More specifically, the three single-stranded oligonucleotide probes (P1, P2 and P3), designed to recognize specific sequences of SARS-Cov2 RNA, were immobilized on a gold electrode surface by incubation for 4 hours (FIG. 1, step (a)). A thiol (HSC₆H₁₂OH) is, in turn, immobilized on the surface of the gold electrode by overnight incubation of the electrode, using a concentration of 10 μM thiol (FIG. 1, step (b)) in order to minimize adsorption and thus non-specific signal. The electrode, on the surface of which the probes and thiol have been immobilized, is incubated over a period between 3 and 4 hours, at a temperature of 50° C., with a sample containing the RNA genome of SARS-Cov2, thus enabling the recognition of the genome by the probes and its hybridization with them, thus forming a supramolecular complex (FIG. 1, step (c)).

Following the hybridization, the electrode was incubated for 2 hours with an electrochemiluminescent intercalating agent, in particular, 14 μM [Ru(phen)₂dppz]²⁺ (FIG. 1, step (d)).

[Ru(phen)₂dppz]²⁺ shows intense luminescence at 600-650 nm when it is intercalated with a double-stranded nucleic acid, while in aqueous solution the emission is drastically quenched, increasing even more the signal-to-noise ratio. Therefore, once [Ru(phen)₂dppz]²⁺ was intercalated into the genome anchored on the electrode surface, the light emission at 600-650 nm was recorded at −0.8 V (compared with the Ag/AgCl reference electrode), i.e., upon the reduction of the S₂O₈²⁻ coreagent that triggers the ECL process.

After the optimization of the generation conditions of the ECL signal, experiments were performed by using both an analytical synthetic genome and the genome extracted from real samples.

### Example 2—Detection of the Synthetic Genome of SARS-Cov2

The system was initially tested with control viral genome of SARS-CoV-2 at different concentrations. The whole lyophilized pathogen (AMPLIRUN Total SARS-CoV-2 Control, Vircell Microbiologist) is from purified viral particles obtained in VERO E6 cells. The virus is diluted in the inactivating viral transport medium (VTM) containing cells obtained from human epithelial cell lines, making the control sample non-infectious and mimicking a real sample. The concentration of the standard used (10,000-25,000 copies/vial) was controlled by the supplier, using the real-time PCR (RT-PCR) method, and was validated by droplet digital PCR (ddPCR). The control genome was prepared according to the manufacturer's manual except the volume of H₂O used to reconstitute the sample—reduced to 240 μL. Next, solid-phase extraction was performed to isolate RNA by a method based on spin-column chromatography with the separation matrix of the silicon carbide resin. An extraction kit of the total RNA from Norgen Biotek Corp was used following the manufacturer's instructions. In summary, the protocol is comprised of the following steps:
1) cell lysis
2) attachment of RNA to the resin of the column
3) removal of the proteins and contaminating impurities
4) elution of total purified RNA

To extract the control genome, 240 μL total volume of the reconstituted sample was used as input and 50 μL of Elution Solution A was used as final elution volume.

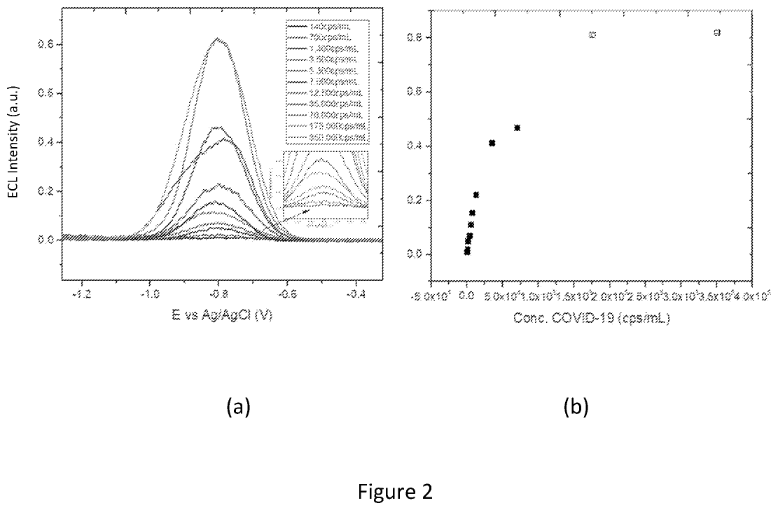

The extracted RNA was quantified by using the previously described method. FIG. 2a depicts the analytical signal as a function of the potential applied over a wide concentration range. FIG. 2b shows the intensity as a function of the concentration of synthetic genome of SARS-Cov2 (COVID 19) and allows the quantification of the limit of the technique at 0.7 cps μL⁻¹.

### Example 3—Detection of the Genome of SARS-Cov2 from Real Samples

The system was thus tested on real samples by using the column extraction method previously described. Simultaneously with our device, the viral load was quantified by real-time quantitative PCR (RT-qPCR, standard method). In real samples, the virus was detected by using a commercial kit (COVID-19 PCR DIATHEVA Detection kit) according to the manufacturer's protocol. The reference kit is a diagnostic test of PCR reverse transcription based on fluorescently labeled probe used to confirm the presence of SARS-CoV-2-RNA by amplification of the RdRp (specific for COVID-19) and E (common for coronaviruses causing severe acute respiratory syndrome) genes. The RNase P gene is also included in the test as an internal positive control, to evaluate the RNA extraction and the presence of PCR inhibitors. Commercial positive and negative controls included in the kit were always included in each RT-PCR assay. The actual sample is identified as positive or negative based on the Ct (cycle threshold) values obtained from the qPCR. The Ct values correspond to the number of PCR cycles needed for the fluorescent signal to cross the threshold, i.e., to exceed the background fluorescence level. The Ct levels are inversely proportional to the amount of nucleic acid in the sample (i.e., the lower the Ct level the greater the amount of RNA). The thresholds applied for positivity/negativity identification are specific to the detection test, and for the kit used, the values are as shown in Table 1.

TABLE 1

Ct values

| RdRp | E | RNase P (control) | Result interpretation |
|---|---|---|---|
| ≤45 | ≤45 | ≤40 | Positive |
| ≤45 | N/A | ≤40 | |
| N/A | ≤45 | ≤40 | Negative |
| N/A | N/A | ≤40 | Negative |
| N/A | N/A | N/A | Failed run |

In Table 2 the qPCR results typically expressed as Ct are shown. It is important to specify that Ct is inversely proportional to viral load and thus to the presence of the virus wherein Ct eater than 45 is an indication of absence of infection)

TABLE 2

| Sample | Covid RT-PCR | Ct | Qubit RNA [ng/μL] |
|---|---|---|---|
| 60937 | Negative | NA | 23.5 |
| 60712 | Positive | 16.44 | 52.2 |
| 60055 | Positive | 19.35 | 78.2 |
| 60635 | Positive | 15.34 | 26.0 |

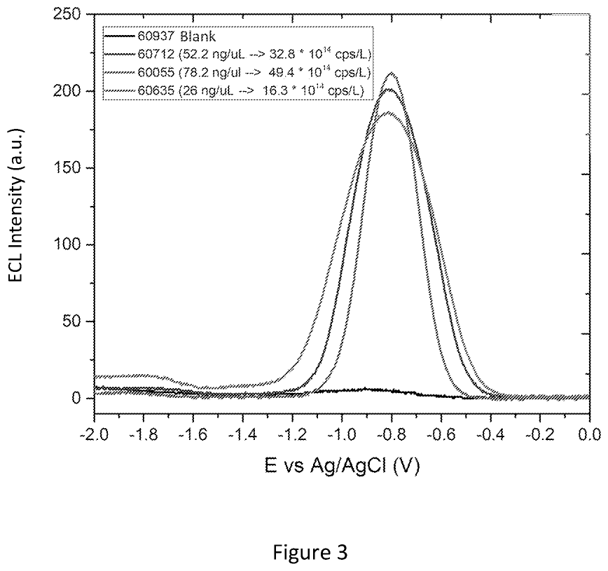

FIG. 3 shows the results obtained by the ECL test.

### Example 4—Detection of the Genome of SARS-Cov2 from Real Samples without Extracting the RNA

The method was tested by evaluating the possibility of eliminating the extraction and replacing it with a simple treatment. In particular, three types of treatment were tested: i) heat-treatment (HT), ii) hydrolysis with proteinase K (PK), and iii) hydrolysis with proteinase and heat-treatment (PK+HT) in combination, iv) without extraction (DIR). The conditions of all treatments are specified in Table 3, below.

TABLE 3

| Treatment | Technical specifications |
|---|---|
| HT | 98° C. for 5 minutes |
| PK | addition of proteinase K [20 mg/ml] in a ratio of 1:10 |
| PK + HT | addition of proteinase K [20 mg/ml] in a ratio of 1:10 |
| | 55° C. for 15 minutes → 98° C. for 5 minutes |
| DIR | direct application of the VTM |

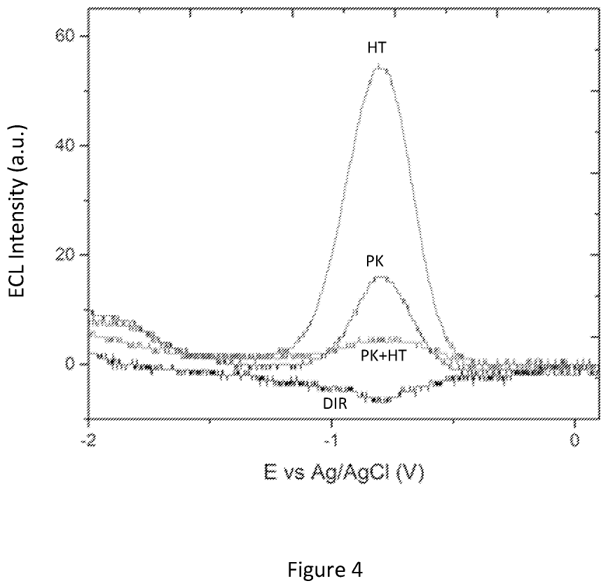

As can be seen from FIG. 4, the best result was obtained by heat-treatment in which the sample positive for viral infection (confirmed by standard extraction and RT-PCR) demonstrates no amplification in PCR (result is considered as negative) while it shows a signal by using the proposed method.

The test is considered “positive” in the presence of a signal produced by both the target sequence and the control.

The test is considered “negative” in the absence of a signal produced by the target sequence but in the presence of a signal produced by the control.

The test is considered “failed” in the absence of a signal produced by either the target sequence or the control.

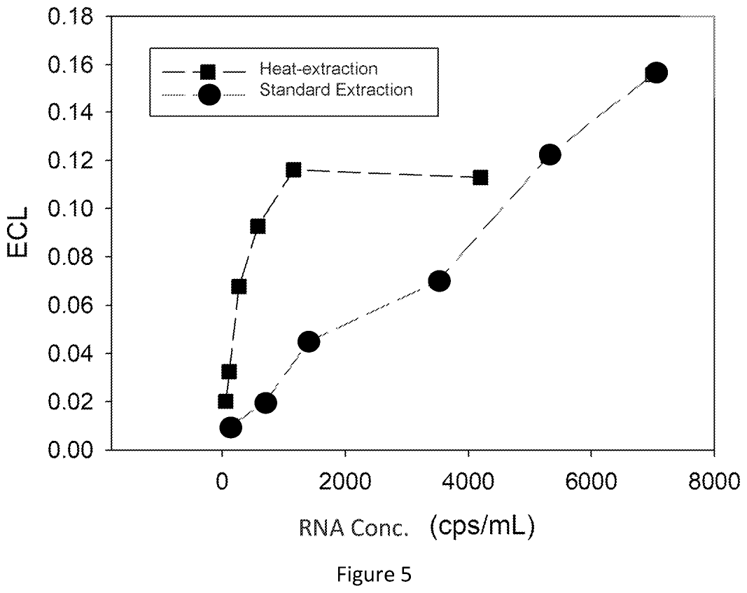

FIG. 5 shows a comparison between the results obtained by heat treating the sample and those obtained by extracting the nucleic acid by known techniques.

As can be seen from FIG. 5, the signal, as a function of the concentration of synthetic genome of SARS-Cov2 (COVID 19), obtained by heat-treatment, is higher than the signal obtained by conventional extraction.

As also highlighted by the Experimental Section discussed above, the present invention has a number of advantages over what is known in the art, in the field of nucleic acid detection, particularly whole genomes (preferably of pathogens such as viruses and bacteria), for analytical and in particular diagnostic purposes.

For example, the process of the invention allows molecular diagnosis (for example, the so-called swab) of infections, for example of COVID, without amplification of genetic material.

Therefore, the process of the invention results in an integrated extraction-detection system, enabling, for example, the rapid and direct molecular determination of infections.

The process of the invention shows high analytical performance (high signal-to-noise ratio, the limit of detection (LoD) is 0.4 cps μL⁻¹ on a standard sample.

Furthermore, the process of the invention is versatile and applicable to different pathogens, including RNA, which can be detected during the same analytical process (for example, determining the cause of a particular pneumonia with the possibility of implementing rapid screening tests thanks to the inherent advantages of the points-of-care (POCs)).

1. A process for detecting an analyte in an isolated sample, wherein said analyte is a target nucleic acid, comprising the steps of:
a) providing an isolated sample comprising at least one nucleic acid;
b) heat-treating said isolated sample thus obtaining a heat-treated sample;
c) contacting said heat-treated sample with at least two single-stranded nucleic acid probes, each of said at least two single-stranded nucleic acid probes being complementary to a corresponding portion of said target nucleic acid;
d) adding an active luminescent luminophore;
e) determining a luminescence signal generated by said active luminophore.

2. The process according to claim 1, characterized in that said isolated sample is treated at a temperature between 60° C. and 99° C. over a period between 1 min and 20 min and subsequently cooled.

3. The process according to claim 2, characterized in that said isolated sample is treated at a temperature between 95° C. and 98° C. over a period between 4 min and 7 min and subsequently cooled.

4. The process according to claim 1, characterized in that said target nucleic acid is DNA or RNA.

5. The process according to claim 4, characterized in that said target nucleic acid is RNA.

6. The process according to claim 1, characterized in that said luminescence signal is a photoluminescence, chemiluminescence, thermochemiluminescence or electrochemiluminescence (ECL) signal.

7. The process according to claim 6, characterized in that said luminescence signal is an electrochemiluminescence (ECL) signal.

8. The process according to claim 1, characterized in that said target nucleic acid is the whole genome of an organism.

9. The process according to claim 8, characterized in that said whole target genome is single stranded (ss-) or double stranded (ds).

10. The process according to claim 8, characterized in that said target genome is a sequence of the whole genome or a single-stranded (ss-) or double-stranded (ds) transcript thereof.

11. The process according to claim 1, characterized in that said single-stranded nucleic acid probes are DNA, RNA or PNA.

12. The process according to claim 7, characterized in that said single-stranded nucleic acid probes are immobilized at an electrode.

13. The process according to claim 12, characterized in that said single-stranded nucleic acid probes are immobilized at an electrode by a surface linker selected from HS—(CH₂)₆—, (OR)₃—Si—(CH₂)₆—.

14. The process according to claim 7, characterized in that said active luminophore is a Ru(II), Ir(III), Re(I), or Os(II) coordination complex or structures derivatized by said complexes.

15. The process according to claim 8, characterized in that said organism is a virus, or a bacterium, or a parasite or a eukaryotic cell.

16. The process according to claim 15, characterized in that said organism is a SARS-Cov2 virus.

17. The process according to claim 16, characterized in that said single-stranded nucleic acid probes comprise at least the sequence GACGTCTAAACCTACTAAAGAGG (SEQ. ID. NO. 1), the sequence CCTTGTGTGGTCTGCATGAGTTTAG (SEQ. ID. NO. 2) and the sequence TAACGTTGTTAGGTACTCGTCACGACTGAG (SEQ. ID. NO. 3).

18. The process according to claim 7, characterized in that said active luminophore is [Ru(bpy)₂dppz]²⁺ or [Ru(phen)₂dppz]²⁺, wherein phen=1,10-Phenanthroline, bpy=2,2′-bipyridine, e dppz=dipyrido[3,2-a:2′,3′-c]phenazine.

19. The process according to claim 7, characterized in that said luminescence signal is generated electrochemically with sacrificial co-reagents.

20. The process according to claim 19, characterized in that said sacrificial co-reagent is selected from S₂O₈²⁻ (persulfate ion), S₂O₈²⁻ (oxalate ion), tertiary amines, tri-n-propylamine, 2-(dibutylamino)ethanol, and hydrogen peroxide.

21. A kit for detecting at least one target nucleic acid comprising a working electrode comprising a material suitable for functionalization, a reference electrode, and a counter electrode.

22. Use of the kit of claim 21 for detecting at least one target nucleic acid.
